(19) United States
(12) Patent Application Publication
(10) Pub. No.: US 2023/0021261 A1
(43) Pub. Date: Jan. 19, 2023

(54) THERAPEUTIC COMPOSITION AND ADMINISTRATION METHODS FOR INCREASING OSSEOINTEGRATION WITH DENTAL IMPLANTS, GRAFT MATERIALS AND PRF

(71) Applicants: Erdal ERGUNOL, ANKARA (TR); Altay ULUDAMAR, ANKARA (TR); Aylin SEPICI DINCEL, ANKARA (TR)

(72) Inventors: Erdal ERGUNOL, ANKARA (TR); Altay ULUDAMAR, ANKARA (TR); Aylin SEPICI DINCEL, ANKARA (TR)

(21) Appl. No.: 17/757,353

(22) PCT Filed: Aug. 20, 2020
(86) PCT No.: PCT/TR2020/050731

§ 371 Date: Jun. 14, 2022

(30) Foreign Application Priority Data
Dec. 15, 2019 (TR) 2019/020272

(51) Int. Cl. C07K 16/22; C07K 16/18; C07K 16/28; A61K 39/395; A61K 35/19; A61K 38/36; A61K 38/29; A61K 31/663; A61P 1/02

(52) U.S. Cl. CPC C07K 16/22; C07K 16/18; C07K 16/2875; A61K 39/3955; A61K 35/19; A61K 38/36; A61K 38/29; A61K 31/663; A61P 1/02

(57) ABSTRACT

The composition and method of the invention relate to dental implant or orthognathic implant, graft materials and platelet-rich fibrin (PRF) applications in the field of dental and jaw health. The invention particularly relates to a therapeutic composition comprising at least one anti-sclerostin antibody and at least one anti dickkopf-1 antibody in combination for increasing osseointegration and accelerating healing in dental implantation and bone volume augmentation in filling of insufficient bone sites, and methods of local administration thereof.

### TECHNICAL FIELD

The composition and method of the invention relate to dental implant, graft material and platelet-rich fibrin (PRF) applications in the field of dental and jaw health. The invention particularly relates to a therapeutic composition for increasing osseointegration and accelerating healing in applications of dental implantation and bone volume augmentation in filling of insufficient bone sites (voids), and methods of local administration thereof.

### PRIOR ART

Nowadays, dental implant treatments have become considerably more widespread for replacing missing teeth and increasing patient satisfaction in terms of improved chewing efficiency, better physical health and aesthetics. Upon placement, the survival and clinical performance of a dental implant are dependent basically upon successful bone formation at the implantation site. Besides, bone graft procedures (such as ridge augmentation and sinus lift) may be required in case of insufficient bone volumes.

Apart from the fact that the dental implantation is a long-term treatment and is difficult to keep patients' cooperation, there may be some limitations associated with this application, such as failure to place the implant immediately after all tooth extractions, anatomical restrictions and reduced bone mass and density. In particular, low success rates in patients suffering from diabetes, immunodeficiency and/or insufficient bone volume are among the most common and important problems.

The average healing and waiting period for dental implants is 2-4 months, and even longer once bone grafting is required before implantation for patients having a bone loss or inadequate bone mass/volume.

Osseointegration of dental implants as well as graft materials and platelet-rich fibrins (PRF) to be used as such is the key factor in the determination of implantation success. Failure in osseointegration, that is, inadequate integration of the implants and thereby loosening over time from jaw bone tissues may require repeating the surgical procedures, which is considered as a failure costing a patient a lot, i.e. money, time, pain and trouble.

Various methods have been disclosed to improve osseointegration within the said context, i.e. manufacturing dental implants by using different materials such as titanium, zirconium, ceramics; roughening their surfaces via related surface modification techniques such as sand blasting or acid-etching; using therapeutic compositions coated with hydroxyapatite, hyaluronic acid and bisphosphonate (zoledronic acid) in order to eliminate endosseous dental defects and to increase bone regeneration; and using PRF alone or in combination with graft materials, which is obtained from the patient's own blood in order to increase bone and soft tissue regeneration and enhancement without causing inflammatory reactions.

In particular, it is known that systemic administration of anti-sclerostin antibody around dental implants increases osseointegration and bone regeneration. For instance, WO2009047356A1 discloses compositions and methods for the use of antibodies against sclerostin. Similarly, U.S. Pat. No. 9,657,090 mentions a method for the treatment of alveolar bone loss through the use of anti-sclerostin antibody. Similar teachings are also available for the anti dickkopf-1 antibody. EP2681242 discloses sclerostin and DKK-1 bispecific binding agents as a single molecular entity against bone disorders. WO2019213285A1 discloses methods of promoting local bone formation, hair growth or wound healing by administering a variety of autologous body materials in combination with an agent that promotes Wnt signaling (e.g., an anti-DKK1 antibody).

Despite these administrations apparently contribute to the success of the bone-implant integration, there is no single outstanding method for achieving the best practice particularly on dental implantation and therefore further new approaches are required for improving and accelerating the osseointegration of dental implants. Because of the nature of this treatment method, a higher risk for complications and failures might be expected, as still-pending problems in the present clinical practice; for instance, repeated subcutaneous administration of therapeutic compositions and thus probable jaw bone tissue injuries, allergic and necrotic dosages, or various possible side effects due to non-local systematic administrations. In particular, it is likely that overqualified and excess bone formation may exist after dental implant treatment and result in pathological fractures.

### OBJECTS OF THE INVENTION

The primary object of the present invention is to increase osseointegration in dental implant, graft material and platelet-rich fibrin (PRF) applications in the field of dental and jaw (orthognathic) surgery and health, building up locally rapid and healthy bone formation, so that the desired/ideal level of bone quality is achieved fast before the conventional treatment period.

Another object of the invention is to eliminate the clinical problems encountered in dental implant, graft material and PRF applications, such as anatomical and systemic restrictions and long-lasting healing period.

It is also an object of the invention to provide satisfactory solutions to patients suffering from systemic or local disorders such as diabetes, immunodeficiency, insufficient bone volume, in dental implant, graft material and PRF applications.

Another object of the invention is to prevent patients from being exposed to subcutaneous injections and systemic drug administration that may negatively affect the overall metabolism with various repeated dosing regimens, in dental implant, graft material and PRF applications.

### BRIEF DESCRIPTION OF THE INVENTION

According to the present invention, it has now been discovered that osseointegration is increased and a rapid and healthy quality bone formation is achieved by administering “locally” a combination of at least one anti-sclerostin antibody (SCL-AB) and at least one anti dickkopf-1 antibody (DKK1-AB), in dental implant or orthognathic implant, graft material, and platelet-rich fibrin (PRF) applications.

Compared to SCL-AB or DKK1-AB administration alone, local administration in combination thereof according to the invention promotes bone integration and regeneration beyond expectations.

Within this scope, the therapeutic composition and method of the invention can be used in order to reduce the time required for osseointegration of the dental implant, graft material and/or PRF before or after placement, to accelerate the healing and treatment process, to prevent implant rejection or failure, and to promote the formation and growth of healthy quality bone.

Any method of local administration known in the prior art may be selected in the application of the composition of the invention, comprising SCL-AB and DKK1-AB, together with the graft material and/or PRF, to the target region, namely, to the patient's diseased gum area, problematic dental and/or periodontal tissues.

In a preferred local use, a combination of SCL-AB and DKK1-AB is applied by adding them into a graft material and/or platelet-rich fibrin (PRF) and/or by brushing, rubbing or coating to the target region before applying a dental implant, matrix or tampon.

In another use, a combination of SCL-AB and DKK1-AB is applied by spraying, dipping, brushing, dropping, rubbing, preserving in a carrier liquid or coating onto the dental implant surface. The amount to be used may vary depending on the size and surface area of the implant, location, and other related disorders of the patient (such as diabetes, osteoporosis, immune deficiency, insufficient bone volume).

In another use, the composition containing SCL-AB and DKK1-AB is applied to the target area as an additive to the graft material and/or PRF before the implant is placed. The amount to be used may vary depending on PRF and type of bone powder in the graft material, the void volume of the extracted tooth and other related disorders of the patient.

In all local administrations, direct or controlled release application of a carrier, matrix or gel containing SCL-AB and DKK1-AB is also possible. Moreover, controlled release applications may include use of specific formulations in which said antibodies will be released at different rates (in terms of amount and/or speed).

In a preferred application of the invention in order to increase the osseointegration of a dental implant used in the field of dental and jaw health or implants used in orthognathic surgery, and to build up a healthy and rapid quality bone formation, the total amount (by weight) of said SCL-AB and DKK1-AB per unit volume of void of the extracted tooth/teeth of the patient or per unit volume of the implant to be applied is 0.001 to 50.0 mg/mm³, preferably 0.01 to 5 mg/mm³ wherein the ratio of said SCL-AB to said DKK1-AB ranges from 10% to 99%, preferably 50% to 90% by weight.

In another preferred application, the dental implant is coated with lyophilized SCL-AB and dry DKK1-AB. Here, a total of about 0.005 mg to about 50 mg, preferably about 0.05 mg to about 30 mg, of the antibody combination may be used, wherein the weight ratio of SCL-AB to DKK1-AB may range from 10% to 99%, preferably 50% to 90%. The amount to be covered may vary depending on the size and surface area of the implant, the thickness of the coating, and other related disorders of the patient.

A healthy quality bone formation can be achieved in a short time by applying in appropriate proportions a composition of SCL-AB and DKK1-AB in combination, together with different types of bone grafts and/or PRF, known from the prior art and readily available on the market, to the tooth extraction region, orthognathic surgery areas and/or onto the dental implant. In this way, the implant application can be started immediately after tooth extraction.

Thanks to the therapeutic composition and method of use of the invention, various challenges and problems during clinical practice on the applications of dental and orthognathic implants such as anatomical and systemic restrictions and long-term healing time can be successfully eliminated. Solutions can even be developed for patients having systemic or local disorders, such as diabetes, immune deficiency and/or insufficient bone volume.

The therapeutic composition of the invention having the SCL-AB and DKK1-AB may further comprise one or more bone enhancing therapeutics to be selected from the group consisting of parathyroid hormone, teriparatide, bisphosphonate and RANKL antibody known from the prior art. Besides, a kit comprising the said composition as a filler to the graft material and/or the PRF may also be provided together with a dental implant or orthognathic implant and instructions for use thereof.

### DETAILED DESCRIPTION OF THE INVENTION

Dental Implant

Dental implants are used for fixing artificial teeth or prostheses to the jaw and can be produced from plastic, ceramic, bioglass, metal and alloys, boron or stem cells. The implants for use according to the invention can be selected from long or short prosthetic implants of 2 to 8 mm length, preferably from titanium, zirconium, ceramic or boron, known from the prior art, or patient's own tooth can be replaced again. Short but wide implants have been preferred recently, particularly in cases of anatomical constraints such as maxillary sinus and mental foramen, and in cases where bone loss is high.

In order to further increase osseointegration, well-known methods such as thermal etching, sandblasting and/or laser roughening to the implant surface may also be applied.

Dental Implant Coating

Various methods known from the prior art may be applied in coating the implant surface. For example, a thermally etched and/or sandblasted titanium implant surface can be modified with physiological serum. Again, the titanium implant may be coated with hydroxyapatite by using the sol-gel method and then coated with the composition of the invention via plasma spraying thereon.

Osseointegration

The term osseointegration refers to the integration of the dental implant, graft material and/or platelet-rich fibrin (PRF) with the surrounding bone tissue, especially in the field of dental, maxillofacial and orthognathic surgery and health. It is important to observe healthy and quality bone formation after implantation. Accordingly, the mineral density of the bone around the implant, the volume of the bone, the bone-implant contact area, the torque and force indicators required for the removal of the implant are taken into consideration and are measured by methods and devices frequently used in the prior art. For example, microCT (microcomputer tomography) or histomorphometry may be used for measuring bone mineral density, bone-implant contact area and bone volume.

Graft Material and Platelet Rich Fibrin (PRF)

Long-term success of osseointegrated dental implants depends largely on the quality and quantity of the available bone in the recipient site and gingiva. Pre-prosthetic procedures such as sinus floor elevation, bone base elevation (augmentation) and/or gingival grafting may be required to reconstruct the ideal bone and gingiva, as there may be atrophy in the jaw bones and thereby surrounding tissues following tooth extraction. In particular, bone grafts and PRF may be used as a filler and support in the field of dental and jaw (orthognathic) surgery and health to facilitate bone formation and promote wound healing according to the invention. These grafts and PRF act as a mineral reservoir that induces new bone formation, are biodegradable and exhibits no antigen-antibody reactions. There are many types of grafts according to the origin of bone powder (i.e. bovine, algae, porcine, beta-tricalcium phosphate (β-TCP), synthetic hydroxyapatite) and material structure. PRF, on the other hand, is derived in gel form from the centrifugation of the patient's own blood and then separation thereof following related techniques. Despite heterogeneous grafts are preferred in common, autogenous grafts may be used, as well. The therapeutic composition and method of use of the invention can be applied in both grafting systems.

Antibodies

Local administration of the anti-sclerostin antibody (SCL-AB) and the anti dickkopf-1 antibody (DKK1-AB) in combination is provided in the method and therapeutic composition according to the invention. In this context, (poly)peptides comprising a framework region from an immunoglobulin gene or fragments thereof providing that specifically recognizes and binds epitopes found on sklerostin and DKK1, respectively and separately; and thus all antibodies selected from monoclonal, chimeric, humanized and/or human antibodies can be applied.

For example, the anti-sclerostin antibody may preferably be selected from synthetic peptides (preferably KLH conjugated) between 12-42 amino acids from the N-terminal region of human SOST, ab63097 (Abcam), Romosozumab (AMG785, Amgen), Blosozumab (Eli Lilly), BPS804 (Novartis) and the anti-DKK1 antibody may preferably be selected from synthetic peptides between 68-83 amino acids from the N-terminal region of human DKK1, Boster and the like.

Tests

To investigate and compare experimentally the effects of SCL-AB and DKK1-AB -each alone and in combination-on bone regeneration and dental implant osseointegration, dental implantation together graft material and PRF were applied to rabbits, and then to pigs, after tooth extraction.

For in-vivo tests, thermally acid-etched and sandblasted titanium implants brought in the carrier liquid were selected among the commercially available ones. The composition of the present invention was sprayed thereon and coated by sol-gel method. As the graft material, Synergy® brand granulated bovine bone graft, and PRF obtained from the blood of the subjects were used.

For the first tests with graft material, seven (7) groups were assigned from totally 42 rabbits, and two comparative studies were conducted. After the extraction of two suitable teeth of each subject, the followings were applied to the each extraction sites (void volume), also reported in Table 1 below: empty socket (control) in the first group; selected bone graft in the second group; followed by SCL-AB only and DKK1-AB only, together with the graft material; and in the last three groups, the SCL-AB+DKK1-AB combination of the invention in varying proportions (50%, 75% and 90%, respectively, as the SCL-AB weight ratio in the total combination).

TABLE 1

| # | First Tooth Void (Test 1) | Second Tooth Void (Test 2) |
|---|---|---|
| Group 1 | Empty Socket | PRF |
| | (Control Group 1) | |
| Group 2 | Graft Material | PRF + Graft Material |
| | | (Control Group 2) |
| Group 3 | Graft Material + | PRF + SCL-AB |
| | SCL-AB | |
| Group 4 | Graft Material + | PRF + DKK1-AB |
| | DKK1-AB | |
| Group 5 | Graft Material + | PRF + SCL-AB + |
| | SCL-AB + DKK1-AB [I] | DKK1-AB - I |
| Group 6 | Graft Material + | PRF + SCL-AB+ |
| | SCL-AB + DKK1-AB [II] | DKK1-AB - II |
| Group 7 | Graft Material + | PRF + SCL-AB+ |
| | SCL-AB + DKK1-AB [III] | DKK1-AB - III |

Approximate volume of one extraction site (as the void left after a tooth extraction) was calculated as 8 mm³. The bone graft to be applied to this extraction site is fixed at 86.0 mg. In additive-free use on the one hand, the graft was diluted with 0.150 ml physiological serum. On the other hand, the followings were added separately into the each graft of the same amount: (1) 0.4-0.5 mg/mL SCL-AB solution with a concentration of 0.150 ml (=0.075 mg), (2) 0.010 mg DKK1-AB, and (3) the SCL-AB+DKK1-AB combination, total weight of which did not exceed 0.025 mg, wherein weight percentage of SCL-AB ranged from 50% to 90%.

In the second test study carried out together with the first one, the PRF was applied to the void of second extracted tooth of each rabbit, instead of the graft material. Thanks to the data obtained in both studies, similar tests were repeated in pigs by extracting premolar and molar teeth, opening nests into voids thereof in accordance with implant procedures and placing 3.3 to 4.8×10 mm titanium, zirconium, boron and ceramic implants, separately.

Following the said surgical operations, healing progress and bone formation around dental implants, graft materials and PRF were evaluated periodically according to the region in comparison with control group 1 (empty socket) and control group 2 (graft material and PRF). At the end of 4th, 6th and 8th weeks, subjects were sacrificed for tissue sampling and related measurements were carried out by using micro-computed tomography (microCT), histopathological methods, hematoxylin-eosin staining, histochemical staining, Masson's Trichrome and immunohistochemical staining (osteocalcin, osteopontin, alkaline phosphatase) methods.

Radiological Analysis

Jaw bones were prepared for micro-computed X-ray tomography (Micro-CT, Super Argus PET/CT, Sedecal, Spain) in a way that only bone structure will remain as soon as possible after sacrificing animals, volume assessment of the socket areas was performed via 3D reconstruction modeling of the micro-CT images. The processing of the target images was carried out with standard resolution, 0.12 mm slice thickness, 40 kV and 140 microA, and the crossectional images were separated into segments, and then volume analysis was performed by loading thereof into the analyze program (3D Slicer, 4.11.0 version GitHub, San Francisco) for evaluation.

In accordance with MicroCT analysis, average bone formation results of the first 4-week are reported for each group in Table 2 below. According to these results, new bone formation rates in the group of SCL-AB and DKK1-AB combination (Group 6), were observed as 20% at the end of the first week, 43% in the 2nd week, 70% in the 3rd week, and almost full in the 4th week. This formation was also preserved in the 6th and 8th weeks. Compared to the administration of SCL-AB alone and DKK1-AB alone together with the graft material, the SCL-AB and DKK1-AB combination of the invention achieves an additional bone formation rate of 19% and 63% respectively in the 3rd week, and 12% and 67% respectively in the 4th week and this is therefore considered as an achievement beyond expectations.

TABLE 2

| | 1st | 2nd | 3rd | 4th |
|---|---|---|---|---|
| #/Bone Formation (%) | Week | Week | Week | Week |
| Group 1 (Empty Socket) | 8 | 13 | 18 | 22 |
| Group 2 (Graft_GM) | 11 | 22 | 37 | 41 |
| Group 3 (GM + SCL-AB) | 18 | 35 | 59 | 82 |
| Group 4 (DKK1-AB) | 15 | 32 | 43 | 55 |
| Group 5 (GM + | 18 | 33 | 45 | 66 |
| %50 SCL-AB + %50 DKK1-AB) | | | | |
| Group 6 (GM + | 20 | 43 | 70 | 92 |
| %75 SCL-AB + %25 DKK1-AB) | | | | |
| Group 7 (GM + | 21 | 39 | 67 | 74 |
| %90 SCL-AB + %10 DKK1-AB) | | | | |

Consistent with these findings, the 2-D bone fill percentages within the daily coronal osteotomy sites at the end of 4th week were highest in the said SCL-AB and DKK1-AB combination treatment groups.

Histological and Histomorphometric Analysis

The jaw bones removed for tissue sampling were fixed in 10% buffered formalin solution for 14 days and decalcified for 60 days at room temperature in 10% ethylenediaminetetraacetic acid in phosphate buffered saline solution or using osteosoft/osteomall (sigma). Samples were placed in cassettes, washed under running water for two hours, dehydrated through increasing ethanol, cleaned in xylene, and then embedded in paraffin. Sagittal sections of 3 pm thickness cut by a microtome were examined after they were applied Hematoxylin Eosin, Masson's Trichrome and immunohistochemistry stains (osteocalcin, osteopontin, alkaline phosphatase) and then placed on glass slides.

In the hematoxylin-eosin staining method, the sections taken from the test groups were kept at 60° C. for 60 minutes in an oven, and then they were removed from paraffin via treatment in xylol (or xylenes) for 3×10 minutes. After slides were passed through the decreasingly graded alcohol series (100%, 96%, 90%, 70%), respectively, and washed for 1 minute under running water, they were stained in Harris Hematoxylin for 2 minutes and then washed for 2×2 minutes under running water. They were immersed in 1% ammonia-water mixture and washed again under running water for 1 minute. The slides were kept in Eozin for 2 minutes, passed through increasingly graded alcohol series (70%, 80%, 96%, 100%), taken into xylol for 2×1 minutes and then mounted in Entellan®. In order to perform numerical evaluation of the complete area size in the tissue, data was created for each group by using a 4× objective for statistical application in the whole area. On the images taken, total tissue area in each section and mature bone tissue areas in the said total tissue area were measured. The newly formed bone tissue index was found by proportioning the mature bone tissue area/total tissue area in each section.

In the Histochemical Staining (Masson's Trichrome) method, the sections taken from the test groups were kept in 60° C. for 60 minutes in an oven, and then they were removed from paraffin via treatment in xylol for 3×10 minutes. After slides were passed through the decreasingly graded alcohol series (100%, 96%, 80%, 70%), respectively, and then washed for 1 minute under running water, they were stained in Weigert's Iron Hematoxylin for 5 minutes and washed for 3×1 minutes under running water. They were immersed in picric acid for 5 minutes and washed 3×1 minutes under running water. Slides were stained for 5 minutes in Biebrich Scarlet-Acid Fuchsin solution. They were kept in phosphotungstic/phosphomolybdic acid for 10 minutes. They were kept in Aniline Blue solution for 10 minutes, passed through increasingly graded alcohol series (70%, 80%, 96%, 100%), taken into xylol for 2×1 minutes and then mounted in Entellan® (Masson's Trichrome Stain Kit, Brand: Polysciences, Inc., Catalog Number: 25088-100).

According to the results of histological analysis, the findings of the 4th week are summarized in Table 3 below:

TABLE 3

| Test | Results (4th Week) |
|---|---|
| Empty Socket | Lengthened by showing osteoconductive activity towards the defect |
| | area of the bone tissue. The defect areas are in the form of fibrous |
| | connective tissue, and there are small, mature bone pieces inside. |
| | Osteoblasts located in the periphery of bone tissue fragments were |
| | observed. |
| Graft Material | Bone tissue showed osteoconductive activity towards empty space. It |
| | was observed that the area was mainly covered with fibrous connective |
| | tissue and there was also bone tissue formation. Within the fibrous |
| | connective tissue areas, there are immature bone tissue areas. |
| PRF | Osteoconductive activity exists in bone tissue, growth into empty space. |
| | Large, irregular, mature bone pieces formed throughout the extracting |
| | area. Areas other than bone tissue fragments have fibrous connective |
| | tissue. There is an increase in fibroblastic activity around bone tissue |
| | fragments |
| Greft + SCL-AB | Osteoconductive growth was observed. Large, irregular graft material |
| | areas covering the extraction area, mature and immature bone tissue |
| | fragments and fibrous connective tissue were observed. Significantly |
| | intense osteoblast and osteogenic activities were observed around the |
| | graft material areas and at the periphery of bone tissue fragments. |
| Greft + DKK1-AB | Osteoconductive growth was observed. Large, irregular graft material |
| | areas covering the extraction area, mature and immature bone tissue |
| | fragments and fibrous connective tissue were observed. Osteoblast and |
| | osteogenic activities were partly observed around the graft material |
| | areas and at the periphery of bone tissue fragments. |
| Greft + | Osteoconductive growth is available. Graft material areas were seen as |
| SCL-AB (75%) + | irregular large spaces. Around the graft material throughout the |
| DKK1-AB (25%) | extraction area, mature bone tissue formations in the form of lamellas |
| | and partly osteoid tissue areas were observed. The outside areas are in |
| | the structure of fibrous connective tissue. Osteoblasts are available at |
| | the periphery of bone tissue fragments. |

Immunohistochemical Staining Method (Osteocalcin, Osteopontin, Alkaline Phosphatase)

From bone tissue blocks of each test groups, 3 μm thick sections were taken to place onto the slides. After the sections were kept at 60° C. for 1 hour in an oven, they were removed from paraffin via treatment in xylol for 3×10 minutes. The slides were then passed through the decreasingly graded alcohol series (100%, 96%, 80%, 70%) and rehydrated. Sections were passed through distilled water for 1 minute twice to purify alcohol. To remove the antigen mask, it was applied with 1:10 diluted EDTA Buffer (AP-9004-999 ThermoScientific) via a pressure cooker. After washing with distilled water for 3 minutes, endogenous peroxidase activity was blocked in tissues that were activated with 3% hydrogen peroxide (TA-125-HP ThermoScientific) for 10 minutes. Sections washed with PBS were protein-blocked (TA-125-PBQ ThermoScientific) for 10 minutes. Primary antibodies were incubated in humid environment for 2 hours (Osteocalcin (Abcam ab13420), Osteopontin (MS-1376-P0 LabVision/ThermoScientific), Alkaline Phosphate (Abcam ab95462), Amplifier Quanto (TL-125-QPB ThermoScientific) for 30 minutes. It was left in HRP Polymer Quanto (TL-125-QPH ThermoScientific) for 30 minutes. Carefully washed with PBS at each stage. In order to identify positive cells, (Osteocalcin, Osteopontin, Alkaline Phosphatase) stained with DAB, stained for 30 seconds in Harris Hematoxylin and washed for 2×1 minutes under running water. They were immersed in 1% ammonia-water mixture and washed again under running water for 1 minute. The stained glasses were passed through the increasingly graded alcohol series and kept in xylol for 5 minutes for having transparency after dehydration.

According to the results of histomorphometric analysis, it was observed that the bone-implant contact in this group was approximately 3.0-fold greater than the one in the control group in the same period, and that new bone formation area is measured as 2 mm² at the end of the first week, 4-5 mm² in the 2nd week, 7 mm² in the 3rd week, and 8 mm² in the 4th week. No inflammatory cell reaction was observed in any area.

Likewise, all findings obtained in the second tests performed with PRF application were compatible with the first tests in terms of demonstrating the success of the SCL-AB and DKK1-AB combination in bone formation and quality.

It was observed that the healing process was accelerated between 5-15% with applying PRF instead of graft material.

Moreover, following the teeth extraction of the pigs, the third and fourth tests were performed by placing dental implants with graft material and PRF. All findings obtained in these in-vivo tests are fully compatible with the previous ones. Although titanium and titanium alloys are the most commonly used implants, the interest in all-ceramic zirconia implants is increasing day by day. While titanium and titanium alloy implants have an edge over others, there are signs that similar successful results can also be achieved with other implant systems.

As the patients' expectation is to replace the missing teeth as soon as possible and the demand for satisfaction towards shortening the healing and treatment process and promoting quality bone tissue surrounding the dental implant, even in risky patient groups as in healthy individuals, the significance of the present invention increases apparently.

All these results clearly demonstrate that one-time local administration of the SCL-AB and DKK1-AB combination of the invention improves osseointegration and bone regeneration around dental implants in a healthy, quality and rapid manner. Considering that the earliest healing duration to be expected in the current conventional practice, following tooth extraction or conventional grafting and PRF treatments, in order to place dental implants in humans, is 8-12 weeks, and approximately 3-months more for awaiting the osseointegration thereof, it is a very critical achievement to reduce the said duration to 4-6 weeks in total, i.e. at least 50% less compared to current conventional practice, by applying the composition of the present invention.

1. A method for increasing the osseointegration of a dental implant or orthognathic implant, creating a healthy, quality and rapid bone formation in the field of dental and jaw health, comprising administering locally at least one anti-sclerostin antibody and at least one anti dickkopf-1 antibody to a patient in receipt of said implant, characterized in a total amount of said anti-sclerostin and anti dickkopf-1 antibodies per unit volume of void of extracted tooth of the patient or per unit volume of the implant to be applied is 0.001 to 50.0 mg/mm³, preferably 0.01 to 5 mg/mm³.

2. The method according to claim 1, characterized in that the ratio of said anti-sclerostin antibodies to said anti dickkopf-1 antibodies is 10% to 99%, preferably 50% to 90% by weight.

3. The method according to claim 1, characterized in that said local administration is achieved by coating said implant with said anti-sclerostin and anti dickkopf-1 antibodies.

4. The method according to claim 1, characterized in that said local administration is achieved prior to implantation by adding said anti-sclerostin and anti dickkopf-1 antibodies into a graft material and/or platelet-rich fibrin (PRF).

5. The method according to claim 1, characterized in that said anti-sclerostin and anti dickkopf-1 antibodies are applied locally to the patient's diseased gum area or problematic dental or periodontal tissues thereof.

6. A therapeutic composition for increasing the osseointegration of a dental implant or orthognathic implant, creating a healthy, quality and rapid bone formation in the field of dental and jaw health, comprising at least one anti-sclerostin antibody and at least one anti dickcopf-1 antibody in combination, administered locally to a patient in receipt of said implant, wherein a total amount of said anti-sclerostin and anti dickkopf-1 antibodies per unit volume of void of extracted tooth of the patient or per unit volume of the implant to be applied is 0.001 to 50.0 mg/mm³, preferably 0.01 to 5 mg/mm³.

7. The therapeutic composition according to claim 6, wherein the ratio of said anti-sclerostin antibody to said anti dickkopf-1 antibody is 10% to 99%, preferably 50% to 90% by weight in the said local administration.

8. The therapeutic composition according to claim 6, characterized by further comprising one or more bone enhancing therapeutics to be selected from the group consisting of platelet-rich fibrin (PRF), parathyroid hormone, teriparatide, bisphosphonate and RANKL antibody.

9. The therapeutic composition according to claim 6, wherein the dental implant or orthognathic implant is coated with at least one anti-sclerostin antibody and at least one anti dickkopf-1 antibody.

10. The therapeutic composition according to claim 6, wherein a gel, graft material, platelet-rich fibrin (PRF) or matrix comprises at least one anti-sclerostin antibody and at least one anti dickkopf-1 antibody.

11. A kit comprising at least one anti-sclerostin antibody and at least one anti dickkopf-1 antibody according to the therapeutic composition of claim 6, wherein the composition is used as a filler to the graft material and/or the platelet-rich fibrin (PRF), and the kit further comprises at least one of a dental implant or orthognathic implant and instructions for use.

12. Local use of administering at least one anti-sclerostin antibody and at least one anti dickkopf-1 antibody in preparation of a therapeutic composition for increasing osseointegration and providing a healthy and rapid bone formation, to a patient in need of dental implant or orthognathic implant application and/or increase in bone volume in fields of dental and jaw health, wherein a total amount of said antibody combination is 0.001 to 50.0 mg/mm³, preferably 0.01 to 5 mg/mm³, per unit volume of void of the extracted tooth of the patient or per unit volume of the implant to be applied.

13. The local use according to claim 12, characterized in that the ratio of said anti-sclerostin antibody to said anti dickkopf-1 antibody is 10% to 99%, preferably 50% to 90% by weight.

14. The 1 12, wherein said local administration is achieved prior to implantation by adding said anti-sclerostin and anti dickkopf-1 antibodies into a graft material and/or platelet-rich fibrin (PRF).

15. The local use according to claim 12, wherein said anti-sclerostin and anti dickkopf-1 antibodies are applied locally to the patient's diseased gum area or problematic dental or periodontal tissues thereof.

16. The local use according to claim 12, wherein the therapeutic composition is in the form of a gel, graft material, platelet-rich fibrin (PRF) or matrix.

17. The therapeutic composition according to claim 6, wherein the anti-sclerostin antibody (SCL-AB) is lyophilized and the anti dickcopf-1 antibody (DKK1-AB) is dried.

18. The therapeutic composition according to claim 17, wherein the dental implant is coated with lyophilized SCL-AB and dry DKK1-AB antibodies.

19. The therapeutic composition according to claim 18, wherein a total of about 0.005 mg to about 50 mg, preferably about 0.05 mg to about 30 mg, of the lyophilized SCL-AB antibody and dry DKK1-AB antibody combination may be used, wherein the weight ratio of SCL-AB to DKK1-AB antibody may range from 10% to 99%, preferably 50% to 90%.
